(19) United States
(12) Patent Application Publication
(10) Pub. No.: US 2026/0057959 A1
(43) Pub. Date: Feb. 26, 2026

(54) ENGINEERING GAIN-OF-FUNCTION MUTANTS OF A WW DOMAIN BY DYNAMICS ANALYSIS

(71) Applicants: Jin Lu, San Jose, CA (US); Ismail Can Kazan, Tempe, AZ (US); Sefika Banu Ozkan, Phoenix, AZ (US)

(72) Inventors: Jin Lu, San Jose, CA (US); Ismail Can Kazan, Tempe, AZ (US); Sefika Banu Ozkan, Phoenix, AZ (US)

(21) Appl. No.: 19/306,331

(22) Filed: Aug. 21, 2025

Related U.S. Application Data
(60) Provisional application No. 63/685,587, filed on Aug. 21, 2024.

(51) Int. Cl. G16B 5/00

(52) U.S. Cl. CPC G16B 5/00

(57) ABSTRACT

Provided herein are systems and methods for characterizing a protein variant including using a computer system to: access simulated protein structure data of the protein, in which the simulated protein structure data indicates a structure of the protein variant while unbound; simulate binding dynamics data indicating binding between the simulated protein structure data and a biological substrate of interest; quantify biophysical properties of the simulated protein structure data and binding dynamics data to produce structural analysis and dynamic analysis of the protein variant; relate the structural analysis and dynamic analysis to functional behaviors of the protein variant; generate a report based on the functional behaviors, structural analysis, and dynamic analysis of the protein variant.

### CROSS REFERENCE TO RELATED APPLICATIONS

This application claims priority to U.S. Provisional Application No. 63/685,587, filed on Aug. 21, 2024, which is incorporated herein by reference in its entirety for all purposes.

### STATEMENT REGARDING FEDERALLY SPONSORED RESEARCH OR DEVELOPMENT

This invention was made with government support under 1901709 awarded by the National Science Foundation and R21 CA207832 awarded by the National Institutes of Health. The government has certain rights in the invention.

### SEQUENCE LISTING

In accordance with 37 C.F.R. § 1.831, the present specification makes reference to a Sequence Listing submitted electronically in the form of an XML file (file name: 112624_01530_ST26.xml; size 7,385 bytes; date generated: Sep. 2, 2025). The entire contents of the Sequence Listing are herein incorporated by reference in their entirety, with the intention that, upon publication (including issuance), this incorporated Sequence Listing will be inserted in the published document immediately before the claims.

### FIELD

The field of the invention relates to methods of protein engineering, and relating protein structure, protein binding, and predicted protein function.

### BACKGROUND

There is a need to understand the relationship between protein sequence-structure and protein binding dynamics. One area of interest is relating foldability and function of a protein and characterizing how foldability and binding change based on mutations in the protein sequence.

### SUMMARY

Disclosed herein are apparatus, systems, and methods for characterizing a protein variant. In one aspect, a method of generating a report that provides a functional characterization of a protein variant is provided. The method may include: accessing simulated protein structure data of a protein variant; simulating binding dynamics of the simulated protein structure and a biological substrate of interest; quantifying biophysical properties of the simulated protein structure data and binding dynamics data to produce structural analysis and dynamic analysis of the protein variant; relating the quantified biophysical properties to functional behaviors of the protein variant; and generating a report based on the functional behaviors, the structure analysis, and the dynamic analysis. The report may include a functional characterization of the protein variant, and additional information on key residues in the protein variant.

In another aspect, a method for designing a protein for a desired function is provided. The method may include: modeling unbound conformations of a base protein to determine a dominant conformation; modeling binding between the base protein in the dominant unbound conformation and a biological substrate of interest to identify a docked pose of the base protein and a contact residue of the base protein, in which the contact residue of the base protein is in direct contact with the biological substrate; determining a flexibility profile of the base protein and modeling dynamics of the base protein; generating a plurality of protein variants based on the docked pose, the contact residue, and the flexibility profile of the protein by substituting amino acids at the contact residue; characterizing each protein variant in the plurality of protein variants based on structural analysis and dynamic analysis of the protein variant to produce a functional characterization of the protein variant; and selecting a protein variant of interest from the plurality of protein variants based on a comparison between the functional characterization of the protein variant and the desired function of the protein variant. The method may be executed using a computer system.

### BRIEF DESCRIPTION OF FIGURES

The patent or patent application file contains at least one drawing executed in color. Copies of this patent or patent application publication with color drawing(s) will be provided by the Office upon request and payment of the necessary fee.

The disclosure will hereafter be described with reference to the accompanying drawings, wherein like numerals denote like elements.

FIG. 1 shows docked poses of mutant residues on the N21 background (left). Sequences of the newly designed WW domain variants with mutations are highlighted (right).

FIG. 2 shows models of ligan bound (CC16-N21) and unbound (N21). 10Y (green) in the ligan (-PPxY-motif) interacts with residues 9Y and 18Y (cyan) in CC16-N21. The corresponding positions are H in N21. Sequence alignment of N21, CC16, and CC16-N21 are shown.

FIG. 3 shows dynamic-based design. DFI plots of N21, CC16-N21, and T10H (top) and color-coded ribbon diagrams showing the DFI profile of each position (red, highest, and blue, lowest DFI values). Positions 10 and 16, distal to the binding site, show hinge shifts that may contribute to binding. Residue position 16P is conserved. Mutating T10 to H in N21 restores the DFI profile toward that of CC16-N21, suggesting that T10H could modulate binding dynamics.

FIGS. 4A-4B show structure and thermostability of WW domain variants. (FIG. 4A) CD spectra of WW domain variants at 4° C. (FIG. 4B) Thermal denaturation Tₘ curves monitored at 227 nm (5° C. to 90° C. ramp, rate of 0.3° C./min). [θ]=Mean Residue Ellipticity; Buffer condition: 20 mM NaPO₄, pH 7.0.

FIG. 5 shows SEC chromatogram of mutants at the concentrations used in ITC. Superdex 30 Increase 10/300 GL column was used in AKTApure and calibrated using appropriate standards (inset). Buffer condition: 20 mM NaPO4 at pH 7.0.

FIGS. 6A-6C show CD spectra (top panel) and Tₘ curve (bottom panel) of the variants using (T10H (FIG. 6A), H9Y (FIG. 6B), H9YH19Y (FIG. 6C)) cuvette of different path lengths at different concentrations. Quartz cuvettes with 1 cm (4 μM) and 1 mm (40 μM) path lengths were used to scan the variants. [θ]=Mean Residue Ellipticity. Buffer condition: 20 mM NaPO4 at pH 7.0.

FIGS. 7A-7B show temperature-dependent CD spectra at 10° C. intervals for H9YH19Y (FIG. 7A) and H19Y (FIG. 7B). [θ]=Mean Residue Ellipticity. Buffer condition: 20 mM NaPO4 at pH 7.0.

FIG. 8 shows reverse thermal denaturation for H9YH19Y: similar Tₘ values (within 1.2° C.) support the absence of intermediate species.1 No visible precipitation is observed. [θ]=Mean Residue Ellipticity. Buffer condition: 20 mM NaPO₄ at pH 7.0.

FIGS. 9A-9C show Isothermal Titration Calorimetry (ITC) of the WW domain variants with Group I peptide measured at 4C. (FIG. 9A) H9Y; (FIG. 9B) T10H; and (FIG. 9C) H9YH19Y Buffer condition: 20 mM NaPO₄, pH 7.0; experiments were carried out in duplicate, and blank was subtracted (FIG. 9C).

FIGS. 10A-10C show ITC Duplicated ITC for H19Y (FIG. 10A, FIG. 10B) variants titrated with Group I peptide. Buffer condition: 20 mM NaPO4 at pH 7.0 (FIG. 10C).

FIG. 11 shows docking poses of the WW domain variants showing H-bonding. The docked poses of CC16-N21, H9YH19Y, N21, H9Y, H19Y, and T10H are shown in cartoon representations. Residues making hydrogen bonding interactions with the peptide ligan (gray) are shown in sticks and annotated in the figure. Hydrogen bonds (yellow dashes) between the peptide ligand and the residues of the protein are highlighted. Five hydrogen bonds are observed for CC16-N21 and H9YH19Y H9Y and T10H only make four hydrogen bonds with the peptide ligan. Only one hydrogen bond is identified for the nonbinders N21 and H19Y.

FIGS. 12A-12B show experimental binding data ln(K_{d}) compared with predicted binding score and change in % DFI of binding residues: (FIG. 12A) Correlation with predicted binding scores obtained from Adaptive BP-dock (R=0.94). The double mutant H9YH19Y has the lowest predicted binding energy score and lowest K_{d}. (FIG. 12B). The change in dynamics (Σ(% DFI_{Mutant}−% DFI_{N21})) of binding residues making hydrogen bonds with the peptide correlates with experimental K_{d} (R=0.98). The variant H9YH19Y has the largest change in the negative direction indicating the largest rigidification at the binding site.

FIGS. 13A-13B show the correlation between principal components and binding affinity. (FIG. 13A). Correlation between PC2 and binding affinity: the projections of time series DFI data on the first and second principal components show separation between binders (in red) and non-binders (in blue) on the second principal components score, where binders have Pc2>0 and non-binders have PC2<0. (FIG. 13B). Correlation between PC1 and melting temperature (Tₘ): the plot of PC1 versus Tₘ shows a clearly positive correlation (R=0.79, p=0.11) between them, indicating that PC1 reflects the stability of N21 and its mutants.

FIGS. 14A-14D show MALD-TOF spectrometry of the WW domain variants (H9Y (FIG. 14A), H9YH19Y (FIG. 14B), and T10H (FIG. 14C)) and Group I peptide (FIG. 14D). H9Y and H19Y have the same molecular weight.

FIG. 15 shows an example of a process for generating a report that indicates a functional characterization of a protein variant in accordance with some embodiments of the disclosed subject matter.

FIG. 16 shows an example of a system for generating a report that indicates a functional characterization of a protein in accordance with some embodiments of the disclosed subject matter.

Those of ordinary skill in the art will understand that the methods and systems specifically described herein and illustrated in the accompanying drawings are non-limiting exemplary embodiments and that the scope of the various embodiments of the present disclosure is defined solely by the claims. The features illustrated or described in connection with one exemplary embodiment may be combined with the features of other embodiments. Such modifications and variations are intended to be included within the scope of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments of being practiced or of being carried out in various ways.

In one aspect, the disclosure provides methods and systems of characterizing a protein variant, including accessing simulated protein structure data of the protein variant, simulating binding dynamics indicating binding between the simulated protein structure and a biological substrate of interest, quantifying biophysical properties of the simulated protein structure data and binding dynamics data to produce structural analysis and dynamic analysis of the protein variant, relating the structural analysis and dynamic analysis to functional behaviors of the protein variant, and generating a report based on the functional behaviors, structural analysis, and dynamic analysis. In some embodiments, a computer system may be used to execute the method. In some embodiments, the protein structure data indicates a structure of the protein variant while unbound. In some embodiments, the report includes a functional characterization of the protein variant.

In some embodiments, the dynamic analysis includes identifying hinges and hinge-shift mechanisms in the protein variant. In some embodiments, the method further includes identifying allosteric sites that modulate binding affinity based on the identified hinge-shift mechanisms.

In some embodiments, quantifying the biophysical properties includes at least one of dynamic flexibility index (DFI) analysis, adaptive BP-dock analysis, statistical coupling analysis, or principal component analysis, wherein time series of the protein structure, the binding dynamics, or both the protein structure and binding dynamics, may be evaluated. In some embodiments, the biophysical properties are selected from a group including melting temperature, binding scores, thermostability, and binding assay profile. In some embodiments, the biophysical property is melting temperature. In some embodiments, the biophysical property is binding score. In some embodiments, the biophysical property is thermostability. In some embodiments, the biophysical property is a binding assay profile.

In some embodiments, the protein variant comprises a mutation in a domain of interest, such as a WW protein domain. In some embodiments, the protein variant includes a mutation in a residue of interest in the protein variant.

In some embodiments, the method further includes synthesizing and purifying the protein variant and using experimental methods to further characterize the protein variant.

In some embodiments, the experimental methods include circular dichroism and isothermal titration calorimetry. In some embodiments, the experimental method is circular dichroism. In some embodiments, the experimental method is isothermal titration calorimetry.

In some embodiments, the method includes characterizing a plurality of protein variants.

In some embodiments, each protein variant in the plurality of protein variants is ranked based on its performance for a desired function. In some embodiments, the performance for a desired function is compared relative to a control (e.g., a wild-type protein).

In some embodiments, the functional characterization comprises binding affinity and lowest energy bound poses. In some embodiments, the functional characterization comprises binding affinity. In some embodiments, the functional characterization comprises lowest energy bound poses.

In some embodiments, the report comprises identifying residues in the protein variant that affect the binding dynamics. In some embodiments, the binding dynamics are improved relative to a control (e.g., a wild-type protein).

“Functional behavior” of a protein refers to protein activity such as flexibility, rigidity, binding dynamics, or coupling dynamics.

“Functional characterization” of a protein or protein residue refers to a description that relates protein structure to protein behavior. For instance, a functional characterization can provide an explanation of coupling dynamics between protein residues and/or protein domains. A functional characterization can also predict the impact of mutations on residues or protein domains; these predictions can characterize a potential mutation as beneficial (e.g., improves the function or enzymatic activity of a protein) or as deleterious (e.g., inhibits the function or enzymatic activity of a protein). A potential mutation can also be predicted to be neutral and have no significant effect on protein activity. A functional characterization can relate the characterization of the protein to protein binding to one or more biological substrates. A functional characterization can also relate the characterization of a protein to the evolutionary conservation of a protein.

In another aspect, a method of designing a protein for a desired function is provided herein. The method of designing a protein may include: modeling, using a computer system, unbound conformations of a base protein to determine a dominant conformation; modeling, using the computer system, binding between the base protein in the dominant unbound conformation and a biological substrate of interest to identify a docked pose of the base protein and a contact residue of the base protein, in which the contact residue of the base protein is in direct contact with the biological substrate; determining, using the computer system, a flexibility profile of the base protein and modeling dynamics of the base protein; generating, using the computer system, a plurality of protein variants based on the docked pose, the contact residue, and the flexibility profile of the protein by substituting amino acids at the contact residue; characterizing, using the computer system, each protein variant in the plurality of protein variants based on structural analysis and dynamic analysis of the protein variant to produce a functional characterization of the protein variant; and selecting, using the computer system, a protein variant of interest from the plurality of protein variants based on a comparison between the functional characterization of the protein variant and the desired function of the protein variant.

In some embodiments, the dynamic analysis of each protein variant includes identifying hinges and hinge-shift mechanisms in the protein variant. In some embodiments, the method further includes identifying allosteric sites that modulate binding affinity of the protein variant based on the identified hinge-shift mechanisms.

In some embodiments, the desired function of the protein is at least one of flexibility, binding affinity, or lowest energy bound poses.

In some embodiments, the base protein is based on a WW domain. In some embodiments, the biological substrate is at least one of a peptide, protein, ligand, or drug.

In some embodiments, the method of designing a protein further includes synthesizing and purifying each protein variant, or the selected protein variant, and using experimental methods for further characterization. In some embodiments, the experimental methods include at least one of circular dichroism or isothermal titration calorimetry.

The methods described herein have several advantages compared to comparable techniques. Combining structural and dynamic analysis provides more detailed characterization of a protein. For instance, it allows one to determine whether unbound dynamics play a significant role in binding dynamics of a protein. This can be used to improve characterization of a protein based on structure, dynamics, and function.

One particular benefit of the methods described herein is identifying hinges in a protein and characterizing hinge-shift mechanisms. Hinge-shift mechanisms account for increases in flexibility in some sites and compensation by rigidification at other distal sites. Analyzing hinge-shift mechanisms can be used to identify allosteric sites which modulate binding affinity through altering dynamics. In addition, characterizing hinge-shift mechanisms provides detailed insight into the overall flexibility of a protein, as well as general function of a protein.

Furthermore, the methods described herein can be used to improve approaches to design proteins. For instance, one can design proteins using both structure-based and dynamics-based techniques. Structure-based techniques provide insights into which residues in a protein bind with a protein of interest and may be particularly sensitive to mutations. Dynamic-based design allows one to recognize hinge positions which may play a role in the overall flexibility of a protein, as well as aid in identifying specific residues that play a role in flexibility. Determining a dynamic profile of a protein variant can be used to compare a protein to a wild-type protein and determine whether the variant replicates the dynamics of the wild-type protein.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” when used herein in reference to a value, refers to a value that is similar, in context to the referenced value. In general, persons of ordinary skill in the art, familiar with the context, will appreciate the relevant degree of variance encompassed by “about”, “approximately”, “substantially”, and “significantly” in that context. If there are uses of the term which are not clear to persons of ordinary skill in the art given the context in which it is used, “about” and “approximately” may encompass a range of values within plus or minus 10% of the particular value and “substantially” and “significantly” may encompass a range of values more than plus or minus 10% of the particular value.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include the standard canonical amino acid residues as well as nonstandard or unnatural amino acid residues contained in the group comprising homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, ufa hor, phosphono, phosphine, heterocyclic, enone, imine, aldehyde, hydroxylamine, keto, or amino substituted amino acid, or a combination thereof, an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids. In some embodiments, a peptide as contemplated herein may include no more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 amino acids. A polypeptide, also referred to as a protein, is typically a length of >100 amino acids (Garrett & Grisham, Biochemistry, 2nd edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

The terms “specifically binds”, or “binding specificity” are used alternatively and in relation to an antibody, refers to the ability of the antibody to form one or more noncovalent bonds with an epitope or antigen via the antibody variable domains. Specificity can be characterized by an antibody-antigen affinity, e.g. as characterized by a dissociation constant (KD) of <100 nM, 10 nM, <1 nM, <0.1 nM, <0.01 nM, or <0.001 nanomolar (nM) (e.g. 10−8M or less, e.g. from 10⁻⁸M to 10⁻¹³ M, e.g., from 10⁻⁹ M to 10⁻¹³ M).

As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

A “dominant conformation” refers to the most frequently observed or biologically relevant three-dimensional structure that a protein adopts under specific physiological or experimental conditions.

An “allosteric site” in a peptide, polypeptide, or protein refers to a specific region, distinct from its active site, where a molecule can bind and induce a conformational change that affects the protein's activity.

A “hinge” refers to a region with a protein that allows for it to move and adopt a different conformation.

A “hinge-shift” refers to a change in the location of a hinge.

A “ligand” is a molecule that binds to a protein, often forming a complex and triggering a specific physiological response in the protein. Ligands include, but are not limited to, drugs, hormones, other proteins, etc.

A “biological substrate” can be a peptide, a protein, a drug, ligand, or another substrate that a protein or protein domain may bind to.

A “drug” refers to a small-molecule drug, typically comprised of 20 to 100 atoms and having a molecular mass of less than 1000 g/mol or 1 kilodalton [kDa].

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, spectrophotometer, etc.

Proteins can be engineered by using conventional techniques such as, for example, synthesis by recombinant techniques or chemical synthesis. Techniques for synthesizing proteins are well known and described in the art

### Example 1: Engineering Gain-of-Function Mutants of a WW Domain by Dynamics and Structural Analysis

Proteins gain optimal fitness such as foldability and function through evolutionary selection. However, classical studies have found that evolutionarily designed protein sequences alone cannot guarantee foldability, or at least not without considering local contacts associated with the initial folding steps. We previously showed that foldability and function can be restored by removing frustration in the folding energy landscape of a model WW domain protein, CC16, which was designed based on Statistical Coupling Analysis (SCA). Substitutions ensuring the formation of five local contacts identified as “on-path” were selected using the closest homolog native folded sequence, N21. Surprisingly, the resulting sequence, CC16-N21, bound to Group I peptides, while N21 did not. Here, we identified single-point mutations that enable N21 to bind a Group I peptide ligand through structure and dynamic-based computational design. Comparison of the docked position of the CC16-N21/ligand complex with the N21 structure showed that residues at positions 9 and 19 are important for peptide binding, whereas the dynamic profiles identified position 10 as allosterically coupled to the binding site and exhibiting different dynamics between N21 and CC16-N21. We found that swapping these positions in N21 with matched residues from CC16-N21 recovers nature-like binding affinity to N21. This study validates the use of dynamic profiles as guiding principles for affecting the binding affinity of small proteins.

### Introduction

Protein sequences encode the necessary information for folding and function and are optimized through evolutionary pressure specific to the environment (Butler et al., 2018; Jiang et al., 2021; Russ et al., 2020; Socolich et al., 2005). Within a protein family, multiple sequence alignment (MSA) reveals that residues are crucial through conservation (i.e., amino acid position preference) and co-evolution statistics, which can be used to predict mutation effects (Bai et al., 2016; Campitelliet al., 2021; Hopf et al., 2017; Kazan et al., 2022; Modi, Campitelli, et al., 2021; Russ et al., 2005; Voelz et al., 2009).

Several classic folding and binding studies have focused on WW domains, one of the most abundant independently folded protein domains in nature, because of their biological importance in regulating transcription, apoptosis, and ubiquitylation by binding to proline-rich peptides (Chen & Sudol, 1995; Ilsley et al., 2002; Macias et al., 1996; Rotin, 1998; Sudol, 1996; Sudol & Hunter, 2000). A WW domain refers to a modular protein domain that mediates specific interactions with protein ligands. Evolutionary inference methods that incorporate co-evolution and conservation were used successfully to design artificial WW sequences that fold similarly to their natural counterparts. However, a significant proportion approximately two-thirds) of the sequences obtained with this approach failed to fold correctly (Russ et al., 2005; Socolich et al., 2005). In previous work, we showed that non-foldability was due to frustration: the N-terminal β-hairpin turn would not form correctly due to strong non-native local contacts. We restored foldability explicit consideration of the early folding steps, thus reducing frustration (Zou et al., 2021). We identified five contacts that stabilize the nascent β-hairpin, and grafted them from a foldable natural homologous sequence, N21 (SEQ ID NO: 1), to an unfolded, designed sequence, CC16. This newly designed variant, CC16-N21 (SEQ ID NO: 2), folds and binds a Group I proline-rich model ligand with a binding affinity comparable with natural WW domains (K_{d}=71 μM) (Russ et al., 2005; Zou et al., 2021). Surprisingly, the native sequence N21, which shares 58% sequence similarity with CC16-N21, shows no affinity to this peptide ligand, even though it is much more stable than CC16-N21 to thermal denaturation (Tₘ 46.8° C. vs. 22.4° C.) (Zou et al., 2021).

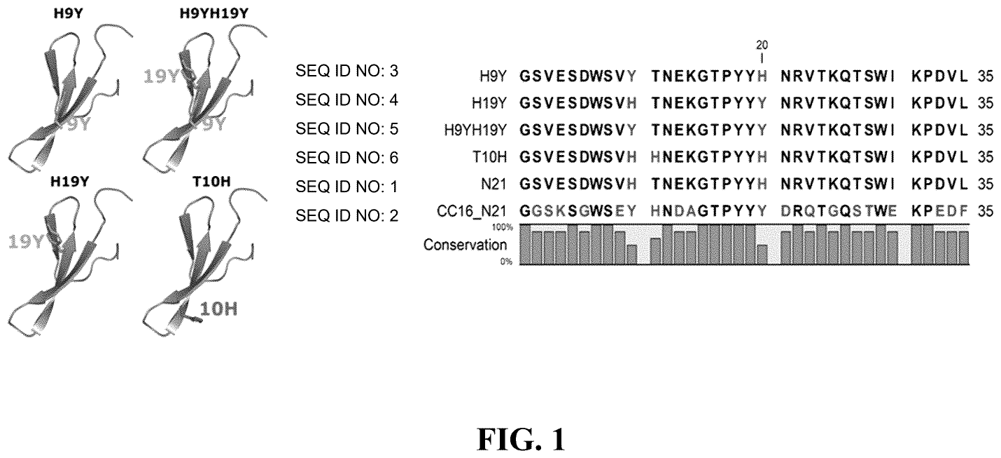

Here, we investigate the balance between folding and binding in the context of WW domains. Our goal is to identify mutations that can modulate the binding affinity of the native N21 sequence to Group I peptides. We performed a comprehensive structural and dynamic analysis on N21 and CC16-N21, using our docking method, Adaptive BP-Dock, to sample the binding trajectories with Group I peptide (EYPPYPPPPYPSG (SEQ ID NO: 7)), and compared the lowest energy bound poses (Bolia & Ozkan, 2016; Bolia, Woodrum, et al., 2014; Kazan et al., 2022). This analysis revealed interactions between the ligand and two tyrosine residues (9Y and 19Y) in CC16-N21 that are absent in the N21 sequence, suggesting that they might be crucial for binding. We introduced tyrosine residues at corresponding positions in the N21 sequence, generating three mutants: H9Y (SEQ ID NO: 3), H19Y (SEQ ID NO: 4), and the double mutant H9YH19Y (SEQ ID NO: 5). See FIG. 1.

In parallel, we explored the conformational differences between N21 and CC16-N21 in the unbound state using dynamic flexibility index (DFI) analysis. DFI is a position-specific metric that computes each residue position's response fluctuation to external perturbations (e.g., random Brownian kick) occurring on the protein chain and is related to conformational entropy per residue position (Nevin Gerek et al., 2013). Positions exhibiting low DFI values (e.g., a DFI percentile value lower than 0.2) are classified as hinges. Hinges are stable locations within the 3D interaction network of a protein, and they do not deviate from their mean when external perturbations occur on the protein. However, due to their extensive interaction network, they can transfer perturbations to the rest of the protein. These rigid hinges can act like joints in a skeleton, mediating the collective motion of the protein, and have been shown to be important for function (Butler et al., 2015; Kolbaba-Kartchner et al., 2021; Kumar et al., 2015; Modi, Risso, et al., 2021; Nevin Gerek et al., 2013). Our previous protein evolution studies showed that proteins modulate function through a hinge-shift mechanism which increases in the flexibility of certain hinges (i.e., hinge losses) are compensated by rigidification at other distal flexible sites (i.e., new hinge formation through mutations during evolution (Kumar et al., 2015; Modi et al., 2018; Modi, Risso, et al., 2021). In the present study, we used this hinge-shift mechanism as a novel conformational dynamics-based computational design approach to find distal sites (i.e., allosteric mutation sites) from the binding residues to modulate binding affinity through altering dynamics. We rationally mold the protein flexibility profile of the N21 based on changes in hinge location upon mutation, then deliberately weigh and alter the dynamics (assessed by DFI profiles) of the designed N21 sequences toward the dynamics of better binder CC16-N21 as done in our other studies (Campitelli et al., 2021; Kumar et al., 2015; Larrimore et al., 2017; Modi, Risso, et al., 2021). We compared the DFI profiles of N21 and CC16-N21: we found a drastic difference in the flexibility of two distal sites (P16, T10), suggesting that they may allosterically modulate binding through alteration of dynamics. Position 16 is a proline in both N21 and CC16-N21, while position 10 is T in N21 and H in CC16-N21. We evaluated this hinge-shift location's contribution to binding by swapping these residues in variant T10H (SEQ ID NO: 6) and examining the resulting binding profile computationally. Finally, all variants designed by either structural or dynamic approaches were expressed and characterized experimentally. These two orthogonal design approaches show that not only the specific interactions of the residues of the binding site, but distal sites can also modulate the binding of the WW domain through dynamic allostery.

### Results and Discussion

Structure-based design of the variants considering the crucial contacts between the N21 and the peptide.

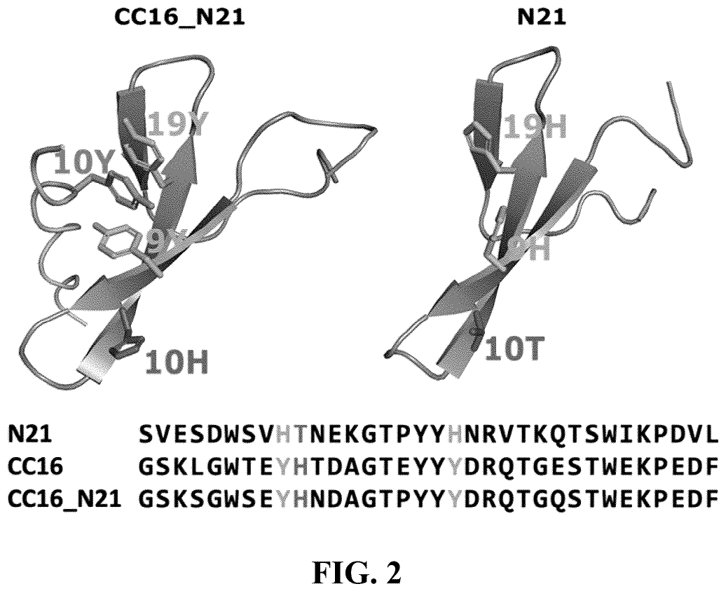

We investigated the molecular interaction governing peptide binding using modeled N21 and CC16-N21 peptide complexes, obtained through homology modeling (Zou et al., 2021). The unbound conformations were subjected to MD simulation and clustered using k-means to gather highly sampled conformations (Kolbaba-Kartchner et al., 2021). The dominant conformation was used as an input representative structure for docking analyses with Adaptive BP-Dock to generate the bound complexes with the Group I peptide. The docked pose with the lowest binding energy score was selected as the bound state (FIG. 2). The docked pose of CC16-N21 shows that tyrosine 9 and 19 are in contact with the peptide ligand. These interactions are missing in N21 bound pose because the sequence of N21 has two histidine at positions 9 and 19. The difference in these interactions is reflected in the computed binding scores of the complexes, −6.91 X-score energy units (XEUs) for N21 and −7.62 XEU for CC16-N21 (Table 1), suggesting that these two residue positions are critical for binding to Group I peptide.

Dynamic-based design of the WW variant utilizing flexibility profiles.

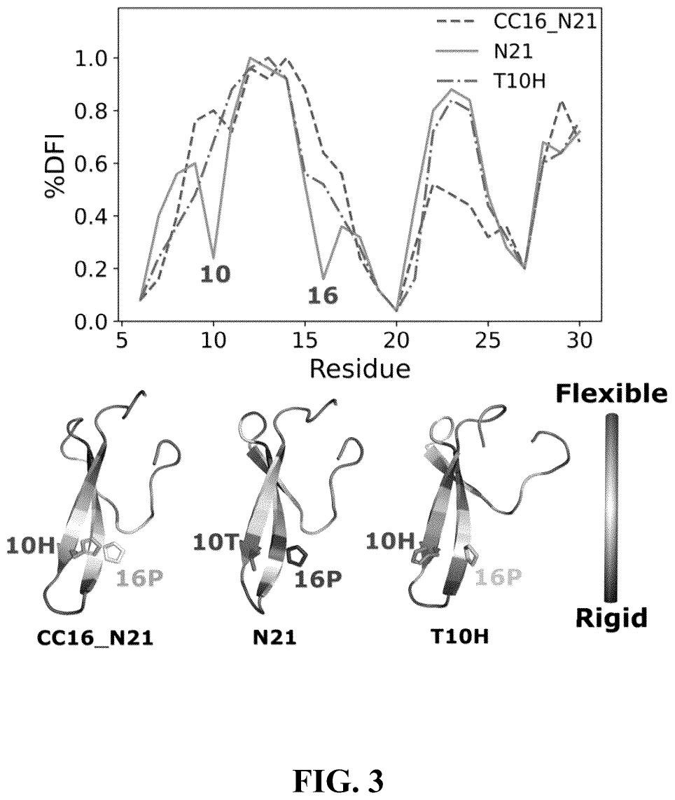

The flexibility profiles of the N21 and CC16-N21 exhibit similar dynamics (FIG. 3), except at positions 10 (histidine in CC16-N21 and threonine in N21) and 16 (proline) in both), which appeared to be hinge-shift positions. Based on other studies suggesting the change in flexibility upon mutation (e.g., hinge-shift mechanism) may impact function, the change in dynamics of these two positions could be responsible for the differences in peptide ligand binding observed between CC16-N21 and N21 (Zou et al., 2021). To investigate this hinge-shift mechanism in detail, we modeled N21-T10H and computed its dynamics. We found that T10H mutation enhances the flexibility at positions 10 and 16: the profile of N21-T10H is similar to that of CC16-N21.

Biophysical characterization of the newly designed variants.

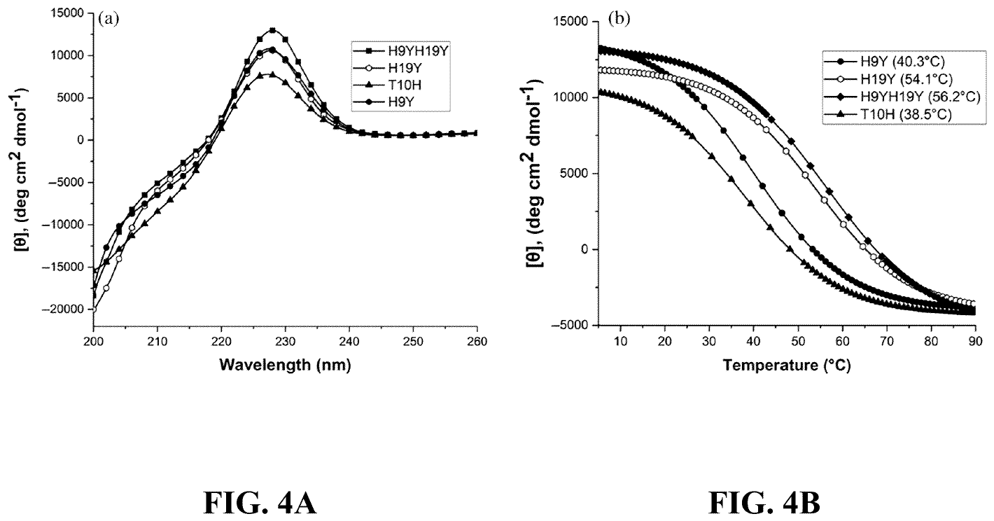

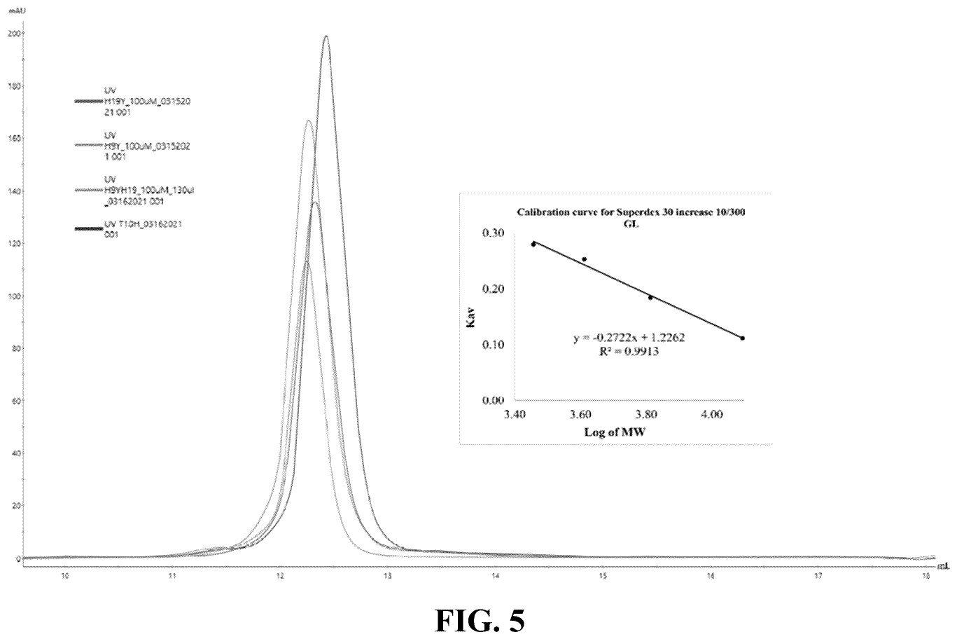

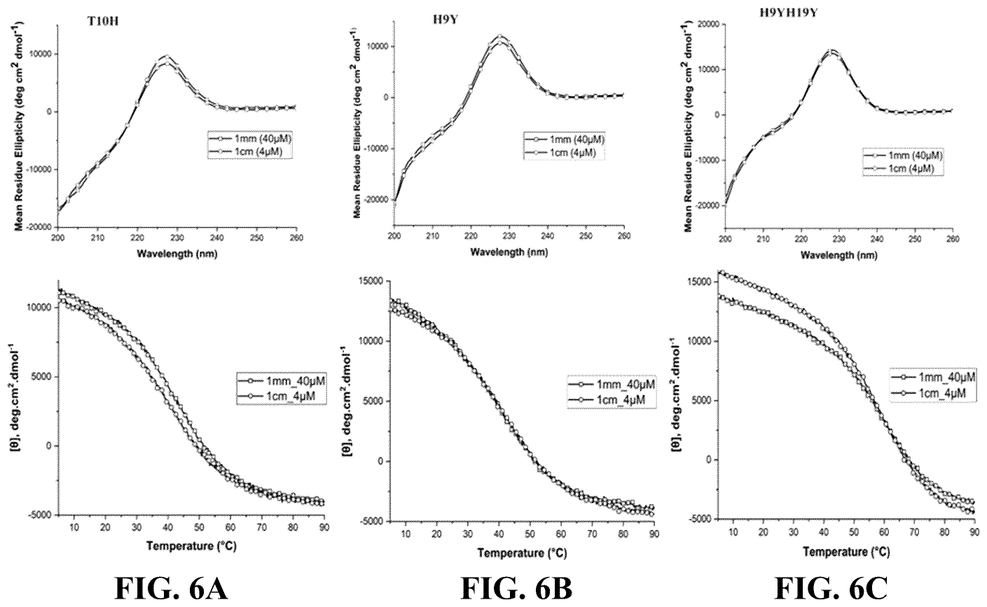

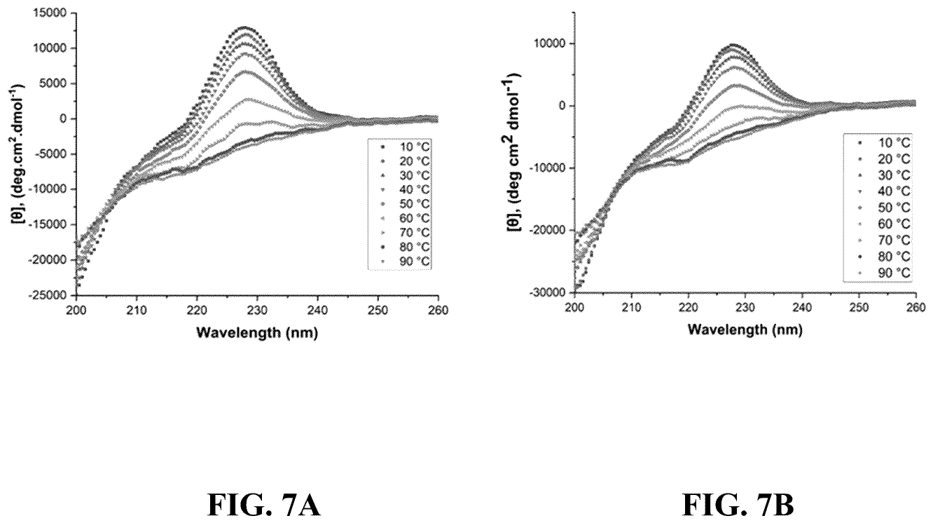

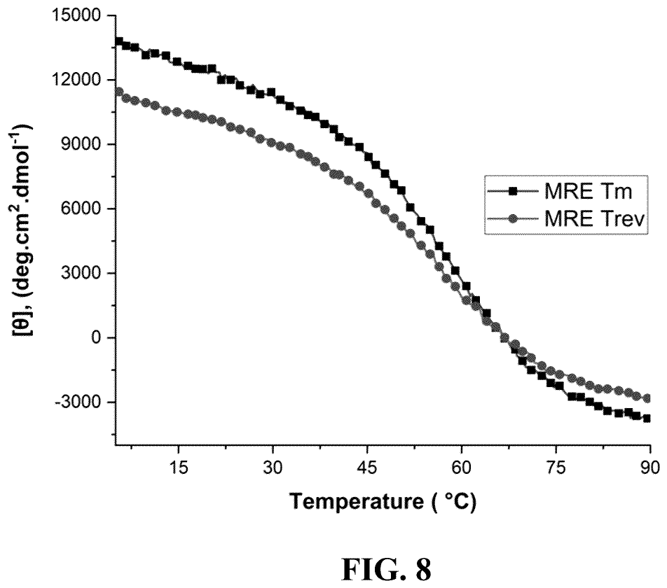

The mutants were prepared by recombinant expression and characterized experimentally. We found that the mutations did not interfere with secondary structure formation, as shown by CD spectroscopy. All mutants, H9Y, H19Y, H9YH19Y, and T10H yielded CD spectra typical of the WW fold and similar to WT N21, with a positive peak centered at 227 nm (FIG. 4A). Thermal denaturation experiments were carried out by monitoring the loss of CD signal at 227 nm in the 5° C.-90° C. range (FIG. 4B); the corresponding Tₘ values are summarized in Table 1. Mutants H19Y (54.1° C.) and H9YH19Y (56.2° C.) are more stable to thermal denaturation than N21 (46.8° C.), while mutations T10H (38.5° C.) and H9Y (40.3° C.) resulted in loss of stability compared to N21; all mutants are within the range of naturally occurring WW sequences (Socolich et al., 2005). The proteins are monomeric at the concentrations used for binding assay as assessed by size exclusion chromatography (SEC) and CD spectroscopy, which show no concentration-dependent variation in Tₘ (FIG. 5 and FIGS. 6A-6C). Additional CD studies support a two-state folding process (FIGS. 7A-7B and FIG. 8).

TABLE 1
Predicted binding scores, thermostability, and binding assay
profile of the WW domain variants are summarized above.

| | | | | | | Binding |
|---|---|---|---|---|---|---|
| WW | | | ΔH | −TΔS | ΔG | energy score |
| variants | Tₘ(° C.) | K_{d}(μM) | (kcal/Mol) | (kcal/Mol) | (kcal/Mol) | (XEU) |
| N21^{a} | 46.8 | — | — | — | — | −6.91 |
| CC16 N21^{b} | 22.4 | 71 ± 4.7 | −3.5 ± 0.2 | −1.3 ± 0.0 | −5.2 ± 0.1 | −7.62 |
| HpY | 40.3 | 86 ± 8.0 | −0.9 ± 0.0 | −4.3 ± 0.0 | −5.2 ± 0.1 | −7.62 |
| H19Y | 54.1 | — | — | — | — | −6.88 |
| H9YH19Y | 56.2 | 50 ± 3.0 | −0.2 ± 0.0 | −5.3 ± 0.0 | −5.5 ± 0.0 | −8.03 |
| T10H | 38.5 | 84 ± 1.0 | −1.2 ± 0.1 | −4.0 ± 0.1 | −5.2 ± 0.1 | −7.63 |

^{a}Naturally occurring variants used as background sequence for this study.
^{b}Variants used in previous studies (Zou et al., 2021) and used for comparison in this study.

Binding to Group I peptide.

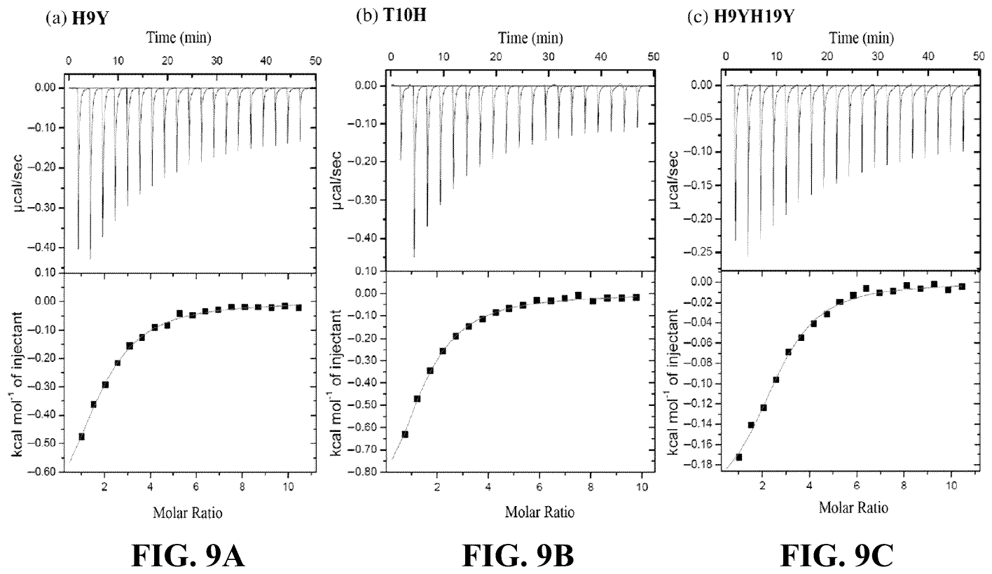

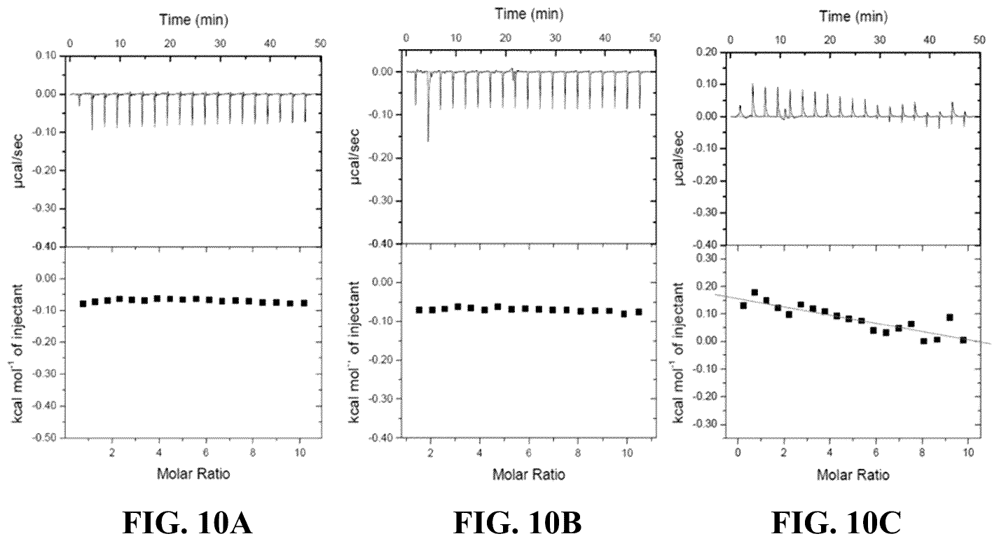

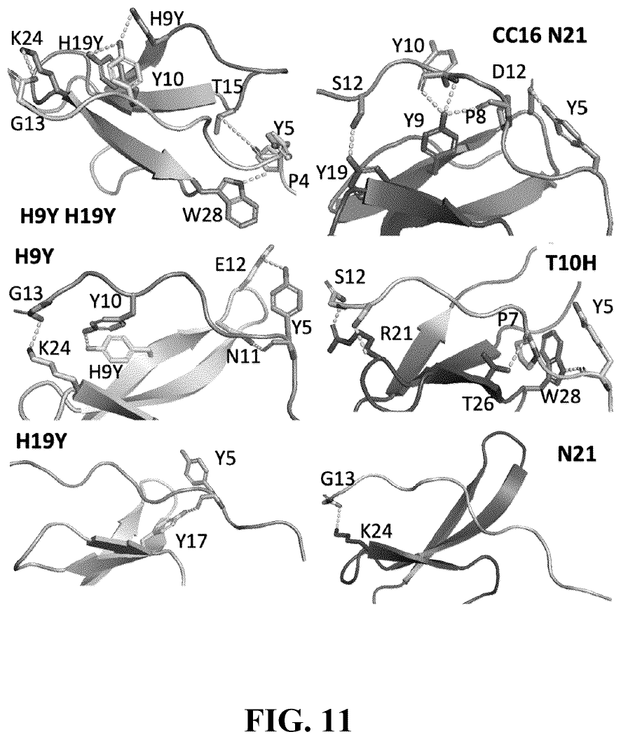

We assessed whether the designed variants bind Group I proline-rich peptide by isothermal titration calorimetry (ITC) titrations and compared the dissociation constant with CC16-N21, for which a K_{d} of 71 μM+4 M had been measured (Table 1) (Zou et al., 2021). We found that T10H and H9Y are comparable to CC16-N21 (K_{d} of 84 μM±1 μM and 86 M+6 μM, respectively). (FIGS. 9A-9B). H9YH19Y displayed improved binding (K_{d}=50 μM+3 μM) (FIG. 9C) while H19Y did not show any binding affinity for the ligand, even at 1 mM concentration (FIGS. 10A-10C). For comparison, naturally occurring WW domains show a wide range of affinity toward Group I peptide ranging from 1 μM to 500 μM (Kato et al., 2004; Russ et al., 2005). We note that H9YH19Y retains the interactions between tyrosine residues and proline residues of the peptide ligand observed in a variety of WW domains (Hu et al., 2004; Kraemer-Pecore et al., 2009; Macias et al., 2002) and captured in the design of the original CC16 sequence based on MSA analysis, as shown in FIG. 11 (Zou et al., 2021).

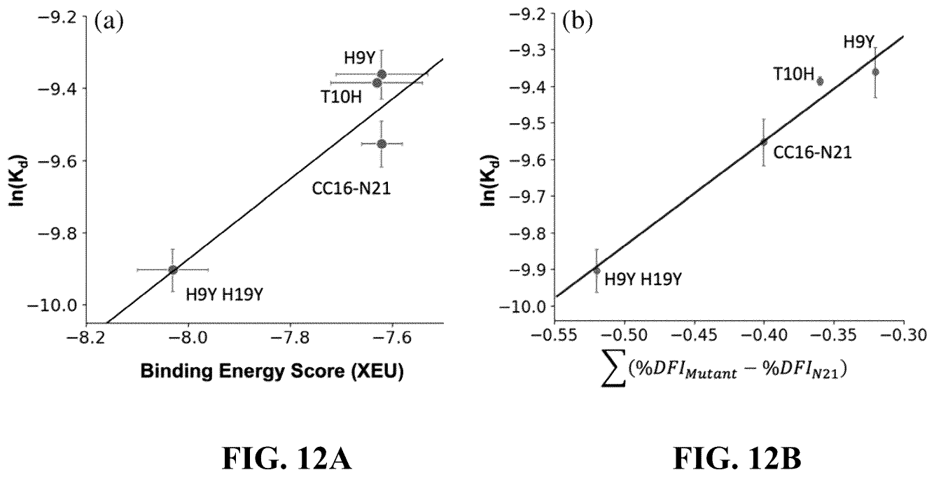

Mirroring our previous observation with the parent sequences CC16-N21 and N21, the experimental binding affinities to Group I peptide do not correlate with thermodynamic stability of the N21 mutant series. Analysis by Adaptive BP-dock and scoring by X-score energy units (XEUs) differentiate binders from non-binders: N21 and H19Y have binding scores higher than −7.00 XEUs while H9Y, T10H, H9YH19Y, and CC16-N21 were clustered below −7.6 XEUs (FIG. 12A) (Bolia & Ozkan, 2016) The experimental binding equilibrium constants, K_{d} (FIG. 12A and Table 1). Additionally, we examined the MD simulations of the unbound variants to understand the dynamics of residues that form the binding surface in WW domains. We utilized the DFI metric to examine the total change in dynamics of these binding residues with respect to the non-binder N21 (FIG. 12B). Variant H9YH19Y had the largest change in the negative direction, indicating the highest level of rigidification compared to the others. In contrast, H19Y exhibited a change in the positive direction, suggesting that the hydrogen bonding residues are more flexible and, therefore, struggle maintaining interactions with the peptide, resulting in a loss of binding.

Previous SCA analysis of the WW domain revealed that eight positions show strong mutual co-evolution with the binding sites (Russ et al., 2005). Some of these positions have no direct interactions with the ligands, suggesting that a distal dynamic allosteric mechanism might be governing the WW domain binding process. We explored whether there are possible allosteric substitutions in CC16-N21 that modulate binding affinity by applying DFI, a position-specific metric that measures the relative flexibility of a residue backbone compared with the rest of the protein. Flexible regions identified by DFI metric tend to have a relatively large residue fluctuation response to a perturbation in other regions, while rigid regions have lower responses. Rigid regions with a DFI score lower than 0.2 are defined as hinges. These hinge sites have critical network of interactions within the 3D fold (Campitelli et al., 2020). They do not exhibit high residue response fluctuations to the perturbations exerted on the protein chain, yet they can transfer the perturbations efficiently to the distal sites of protein, like joints in a skeleton, and play a critical role in modulating the collective motion of a protein. Hinges are critical to protein function: for example, mutations in these positions alter the conformational dynamics profile and correlate to disease-associated mutations in ferritin (Kumar et al., 2015). More broadly, DFI profiles are associated with function and changes in DFI value, particularly in rigid sites, lead to changes in function (Teilum et al., 2009; Xu et al., 2008). Comparative dynamics analysis of ancestral proteins with their corresponding extant homologs revealed that change in DFI profile, particularly compensation of the loss of certain hinge locations by the formation of the new hinge sites called a hinge-shift mechanism, is utilized by nature to manipulate protein function (Modi, Risso, et al., 2021). The DFI analysis of Human Pin1 also showed that substrate binding to the WW domain induces a hinge shift mechanism and enhances the catalytic efficiency (Campitelli et al., 2018).

Hydrogen bonds are important interactions in biological systems, as they contribute to the stability and function of proteins and other biomolecules. Thus, we analyzed the number of hydrogen bond patterns of the docked poses and computed the number of hydrogen bonds formed between the peptide ligand and the binding residues for each mutant (FIG. 11). We found that CC16-N21 and H9YH19Y formed five hydrogen bonds, H9Y and T10H formed four hydrogen bonds, and only one hydrogen bond was identified for the nonbinders N21 and H19Y This analysis also aligns with our dynamics analysis and suggests that the enhanced flexibility of binding residues in N21 and H19Y leads to loss in the formation of hydrogen bonds, thus leading to poor binding affinity.

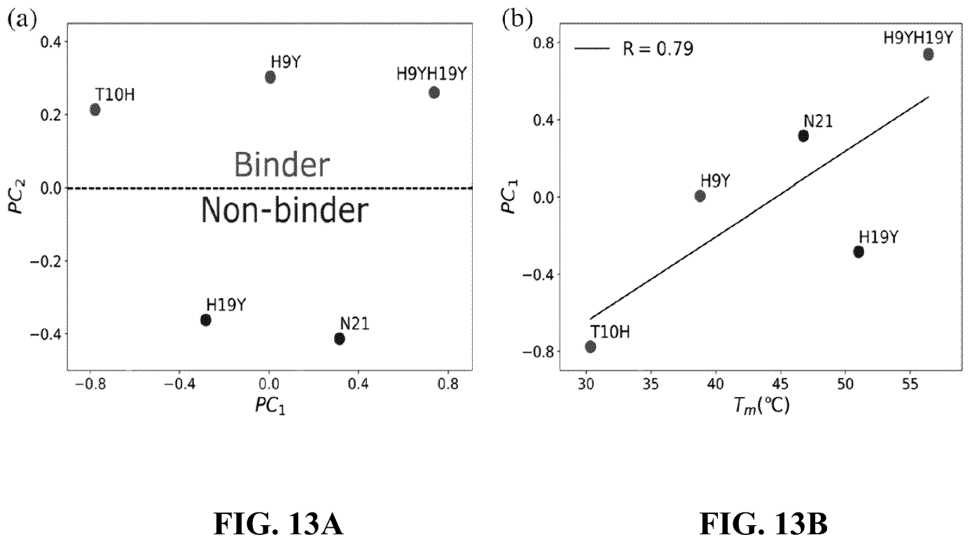

Since our results strongly support that the dynamics of the WW domain play a critical role in its biophysical properties, we further investigated the equilibration and the relaxation of dynamics of variants. We studied % DFI in a sequential manner by creating a time series that carries information on the evolution of the dynamics. We prepared the time series % DFI by averaging 3 adjacent time windows, respectively (0.5 μs-1 μs, 1 μs-1.5 μs, and 1.5 μs-2 μs). As our earlier works (Butler et al., 2015; Kazan et al., 2022; Larrimore et al., 2017; Modi, Campitelli, et al., 2021) highlight that the DFI profiles capture the related function (Butler et al., 2018; Campitelli et al., 2021; Kolbaba-Kartchner et al., 2021; Modi, Risso, et al., 2021; Ose et al., 2022; Stevens et al., 2022; Zou et al., 2015), we cluster these time series of the DFI values of each variant using PCA (see Methods, Principal Component Analysis) to compare their dynamics profiles. The first two principal components are responsible for most of the variance in the mutant DFI profiles. Hence, we utilized these two first principal components to analyze the clustering of the mutants based on their similarity in flexibility profiles. The projection of the data on the first and second principal components shows that the second principal component (PC2) clearly separates binders and non-binders (FIG. 13A). All variants exhibiting binding to Group I peptide have positive PC2 scores, indicating that they have similar flexibility profiles associated with binding dynamics. In contrast, H19Y is clustered with the native N21 with negative PC2 scores, suggesting that their unbound dynamics results in poor binding (FIG. 13A). The first principal component (PC1) captures folding stability: its value is correlated with melting temperature (Tₘ) (FIG. 13B). This analysis differentiates the role of dynamics in modulating protein stability and binding poses and can help explain why H19Y does not bind the ligand.

### Conclusion

We conducted a comparative analysis of the WW domains N21 and CC16-N21 to explore why N21 exhibits poor binding, while its close homolog, artificially designed CC16-N21, showed high affinity to Group I peptides. The peptide-bound structure obtained by adaptive BP-dock highlighted differences in the binding domains between N21 and CC16-N21. Binding in CC16-N21 is mediated by two tyrosine residues (9Y and 19Y) that contact the peptide, whereas in the N21 sequence, the equivalent positions are occupied by histidine. We explored whether unbound dynamics (i.e., the unbound conformational ensemble) played a major role in binding by computing DFI profiles, which provide position-specific metrics related to conformational entropy per site. The comparison of the DFI profiles suggested a hinge-shift point at a distal position 10, which is a histidine in CC16-N21 but a threonine in N21. Based on these observations, we generated four mutants of N21 by substituting these residues: H9Y, H19Y, T10H, and the double mutant H9YH19Y The bound forms were modeled using Adaptive BP-Dock and ranked according to their docking energy scores. The variants were experimentally characterized: all formed secondary structures comparable to N21, although mutations modulated the stability to thermal denaturation. Furthermore, the binding affinities to Group I peptide correlated with the predicted docking energy scores. When we coupled this analysis with the computed DFI values of the positions that formed hydrogen bonds with the peptide, we observed that enhanced flexibility at these binding residue positions correlated with impaired binding in N21 and H19Y Principal component analysis of time-series DFI sheds light on the role of unbound dynamics in governing binding and stability. These results suggest that dynamics govern WW domain binding and that sites that do not directly interact but distally modulate the dynamics of binding may also be crucial and fundamental for binding. We hope that our structure and dynamics-based protein design approach can be used to predict protein binding in general and to study protein-ligand interactions.

### Materials and Methods

### Molecular Dynamics Simulations

Molecular dynamics simulations of the wild-type and mutants were performed using AMBER 20 (Salomon-Ferrer et al., 2013). The mutants were modeled by PyMOL Mutagenesis Wizard (DeLano, 2002). Topology files were prepared based on ff99SB forcefield and the solvation box was modeled by explicit water model TIP3P (Mark & Nilsson, 2001) with a 14 Å minimum distance from the boundary to protein. The systems were neutralized by adding sodium and chloride ions and then minimized with the steepest descent algorithm followed by the conjugate gradient method for 5000 steps. The systems were then heated up to 300 K. Each system was then simulated for 2 μs with 2 fs time-step at constant temperature (300 K) and pressure (1 bar) with Langevin thermostat and barostat.

### Adaptive BP-Dock

Adaptive BP-dock (Bolia & Ozkan, 2016; Kazan et al., 2022) is an iterative docking approach that utilizes perturbation response scanning (PRS) (Atilgan et al., 2010) combined with the RosettaLigand program (version 3.5) (Meiler & Baker, 2006) to model the interactions between the WW domain and the peptide ligand. The induced fit that emerged from the binding event is challenging to model with docking tools with static protein backbone and peptide movement. In Adaptive BP-Dock, we include both the backbone flexibility of the receptor and the ligand. Before each docking step, a new conformation of the protein receptor is calculated based on the residue response fluctuation profile upon force perturbations on the binding pocket residues using PRS.

This approach mimics the peptide ligand's forces acting on the receptor and generates a new conformation that samples binding-induced conformations. This conformation is then docked with the peptide ligand using RosettaLigand. Adaptive BP-dock which includes binding-induced backbone conformational changes improves the modeling of binding interactions and can predict binding scores that capture the binding trends seen in experiments. The predicted binding scores are evaluated by X-score empirical scoring function. X-score energy units (XEUs) have been shown previously to provide a good correlation with experimental results (Bolia, Gerek, & Ozkan, 2014; Bolia & Ozkan, 2016; Wang et al., 2002). Thus, we applied Adaptive BP-dock to each of the most representative clusters sampled during unbound MD simulations. We conducted three separate docking simulations to ensure the binding interactions between the WW domain and peptide ligand are captured accurately.

### Dynamic Flexibility Index

Dynamic flexibility index (Butler et al., 2018; Gerek & Ozkan, 2011; Kazan et al., 2022; Kumar et al., 2015; Larrimore et al., 2017; Modi, Risso, et al., 2021; Ose et al., 2022; Stevens et al., 2022) uses the PRS technique that combines Elastic Network Model (ENM) and Linear Response Theory (LRT) (Nevin Gerek et al., 2013). In PRS, Brownian-like unit forces F are applied sequentially to each residue as perturbations (Atilgan et al., 2010; Kumar et al., 2015). According to LRT, the linear response vector perturbation ΔR due to F is calculated as follows:

$${[ Δ R ]}_{3 N \times 1} = {{[ H ]}_{3 N \times 3 N}^{- 1} [ F ]}_{3 N \times 1}$$

where H⁻¹ is the inverse of the Hessian matrix.

In this work, instead of using Hessian matrix calculated via ENM, we used covariance matrix G for C-alpha atoms calculated from MD trajectories which are proportional to the inverse of the Hessian matrix (H⁻¹). This is because MD provides more precise residue-residue interaction, such as long-range interactions and solvation effects via atomistic fields.

$${[ Δ R ]}_{3 N \times 1} = {{[ G ]}_{3 N \times 3 N} [ F ]}_{3 N \times 1}$$

To compute DFI, we perturbed each residue sequentially by applying random unit forces on each residue. We then generated Perturbation Responses Matrix A as follows.

$${A}_{N \times N} = [ {\vert Δ {R}^{1} \vert}_{1} … {\vert Δ {R}^{N} \vert}_{1} ⋮⋱⋮ {\vert Δ {R}^{1} \vert}_{N} … {\vert Δ {R}^{N} \vert}_{N} ] ,$$

where |ΔR^{j}|ᵢ= [symbol] denotes the average response at position I due to perturbations on j.

This procedure is repeated several times in different directions for each position, to ensure that forces are isotropically sampled. Then the averaged Perturbation Response Matrix A is used to calculate the DFI per residue.

$${DFI}_{i} = \frac{{\sum }_{j = 1}^{N} {\vert Δ {R}^{j} \vert}_{i}}{{\sum }_{i = 1}^{N} {\sum }_{j = 1}^{N} {\vert Δ {R}^{j} \vert}_{i}}$$

This index is often more useful as a percentile since the DFI range varies for different proteins. Therefore, the DFI percentile is calculated as

$$\% {DFI}_{i} = \frac{{n}_{\leq i}}{N}$$

where N is the total number of residues and n_{≤i} is the number of residues with DFI value≤DFIᵢ.

To understand and capture the converged dynamics of the protein system, we calculated time series % DFIᵢ based on MD covariance matrices from three sequential time window: (i) 0.5 μs-1 μs, (ii) 1 μs-1.5 μs, and (iii) 1.5 μs-2 μs. To improve the accuracy, the loose ends of the proteins were excluded.

### Principal Component Analysis

Principal component analysis (PCA) was applied to time series DFI. This dimensionality-reduction method is used to reduce the variables in a high-dimensional dataset while retaining most of the information from the dataset, therefore making the data more interpretable (Jolliffe & Cadima, 2016). For N21 and its mutants, the time series DFI profiles were merged into n×p matrix X where n=5 (total number of DFI profiles) and p=75 (dimension of time series DFI) (Kolbaba-Kartchner et al., 2021). Singular value decomposition of X was conducted as follows:

$${[ X ]}_{n \times p} = {{{[ U ]}_{n \times n} [ Σ ]}_{n \times p} [ V ]}_{p \times p}^{T}$$

Here, U and V are unitary matrices with orthonormal columns which are called left singular vectors and right singular vectors, respectively. Σ is a rectangular diagonal matrix of positive number σᵢ called the singular values of X. σᵢ were arranged, by convention, in a decreasing order of their magnitude and represent the variances in the corresponding left and right singular vectors.

The column vectors of V are called the principal components. They are new variables that are constructed from the initial variables where the first principal component is a direction that maximizes the variance of the projected data, therefore preserves most of the data's variation. Score matrix T is defined as follows:

$${[ T ]}_{n \times p} = {{[ U ]}_{n \times n} [ Σ ]}_{n \times p}$$

Each row vector of T is the projection of the corresponding data vector from matrix X on every principal component. In this study, we utilized the projections of our original data vectors on the first and second principal components and discovered their relations with the protein functions.

### Plasmid Sequencing and Protein Expression

The sequences encoding for the designed WW domain proteins, containing point mutation(s) on N21 native sequence, were ordered from Genscript. All mutants were fused to Maltose Binding Protein (MBP) and cloned in pMAL-c5x vector for expression. Each gene contained an N-terminal poly-histidine tag and the TEV cleavage site ENLYFQG to facilitate purification. The plasmids were transformed via heat shock into competent E. coli BL-21 cells (NEB) and the mix was plated on LB agar plates containing ampicillin overnight at 37° C. Single colonies were used to inoculate 5 mL LB liquid cultures containing ampicillin and were grown overnight at 37° C. shaking at 200 RPM. 10 mL of each culture was transferred to a 2 L flask containing 1 L LB media with ampicillin for growth and expression. The rest of the cells were centrifuged down, and the plasmid DNA was extracted using Promega Wizard® Plus SV Miniprep kits. Sequences were verified using GeneWiz Sanger Sequencing. The 1 L cultures were grown to OD600 of 0.6-0.8 and protein expression was induced by addition of 1 mM IPTG. Proteins were expressed for 6 h at 37° C. shaking at 200 RPM. The total protein yield for these conditions was roughly 20 mg/L.

### Protein Isolation and Purification

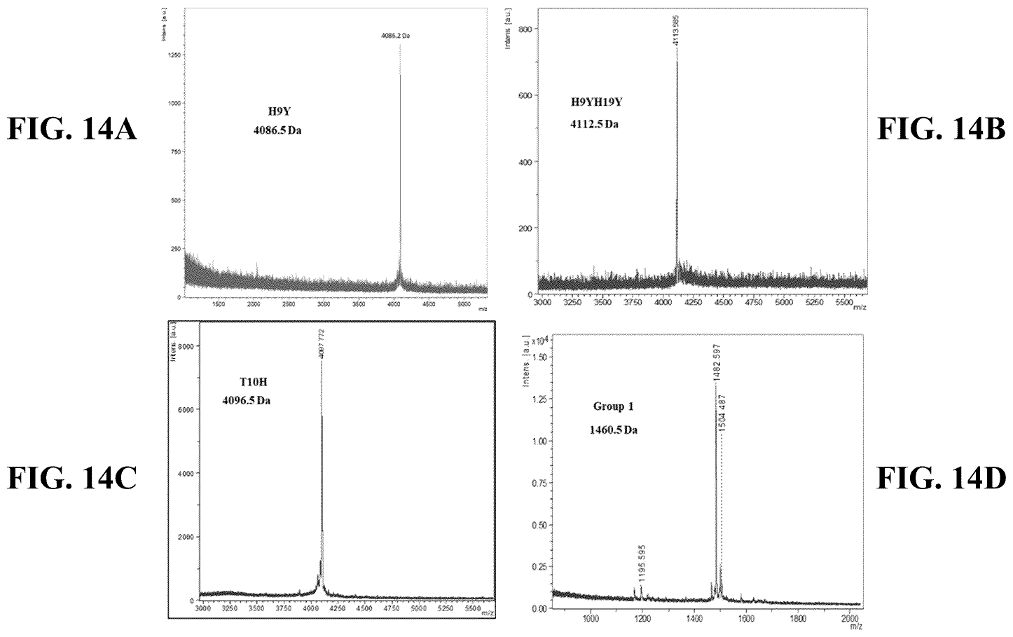

Cells were harvested by centrifugation at 5000 RPM for 20 min and resuspended in 30 mL 20 mM NaPO4 at pH 7.4, 0.5 M NaCl, and 20 mM imidazole buffer. Cells were lysed by sonication for 20 minutes using ON/OFF cycle by 30 s, and then spun down at 5000 RPM, 4 for 1 h. The supernatant was purified on a 5 mL Amersham Bioscience HisTrap column by FPLC (AKTApure). Fractions containing the protein were dialyzed in 20 mM NaPO4 at pH 7.4, 0.5 M NaCl, and 10 mM imidazole at 4° C. The His-tag was cleaved by digesting the proteins with TEV at a ratio of 1:20 TEV to fusion protein, followed by purification by HisTrap column; WW proteins were collected in flowthrough. The proteins were further purified by RPHPLC on a 250×10 mm Phenomenex C18 Semi-prep column by gradient elution starting with 0.01% TFA in water solvent A) to 95% acetonitrile with 0.01% TFA (solvent B). Purified proteins were verified by MALDI (FIGS. 14A-14D) and stored at −20° C. after being lyophilized.

### Group I Peptide Synthesis and Purification

Proline-rich peptide, Group I, was synthesized on a CEM Liberty automated peptide synthesizer using Wang resin and FMOC-protected amino acids. Deprotection conditions: 20% Piperidine, 0.1 M HOBT in DMF. Activation and coupling solutions: 0.5 M HBTU and 2 M DIEA in NMP. After completion of the synthesis, cleavage from resin was accomplished by shaking for 2 h using a cleavage cocktail containing 95% TFA, 2.5% Triisopropylsilane, and 2.5% distilled water. After 2 h, the mixture was filtered, excess TFA was removed, and lyophilized. The crude peptide was purified by RP-HPLC on a 250×10 mm Phenomenex C18 Semiprep column by gradient elution starting with 0.01% TFAin water (solventA) to 95% acetonitrile with 0.01% TFA (solvent B). and verified by MALDI (Bruker).

### Circular Dichroism

Protein stability and folding were assessed by Circular Dichroism (CD) using a JASCO J-815 CD Spectrophotometer (JASCO, Easton, MD). Full scans were measured from 280 nm-200 nm at 5° C. with a 1 cm (or 1 mm) quartz cuvette, at protein concentration of 40 μM in 20 mM NaPO4 buffer at pH 7.4. Spectra were collected in triplicate, averaged, and converted to mean residue ellipticity (Greenfield, 2006).

Denaturation temperature (Tₘ) for all the WW domain peptides was calculated by monitoring ellipticity at 227 nm while increasing temperature from 5° C. to 90° C. at a ramp rate of 0.3° C./min. Data were analyzed to extract Tₘ according to established methods in OriginPro 2018 (Greenfield, 2006).

### Isothermal Titration Calorimetry (ITC)

WW domain and Group I peptides were sent to Sanford-Burnham Medical Research Institute (La Jolla, CA) for ITC using an ITC200 calorimeter from Microcal (North Hampton, MA). In short, aliquots of Group I peptide from a 5 mM stock were titrated into 100 μM WW domain peptides, in 20 mM NaPO4 buffer, pH 7.0. Data was analyzed using standard fitting procedures with a one-binding site model and analyzed using the Origin software package provided by Microcal. Titrations were carried out in duplicates, using phosphate buffer as blank.

### Example 2: Illustrative Embodiments of Presented Methods

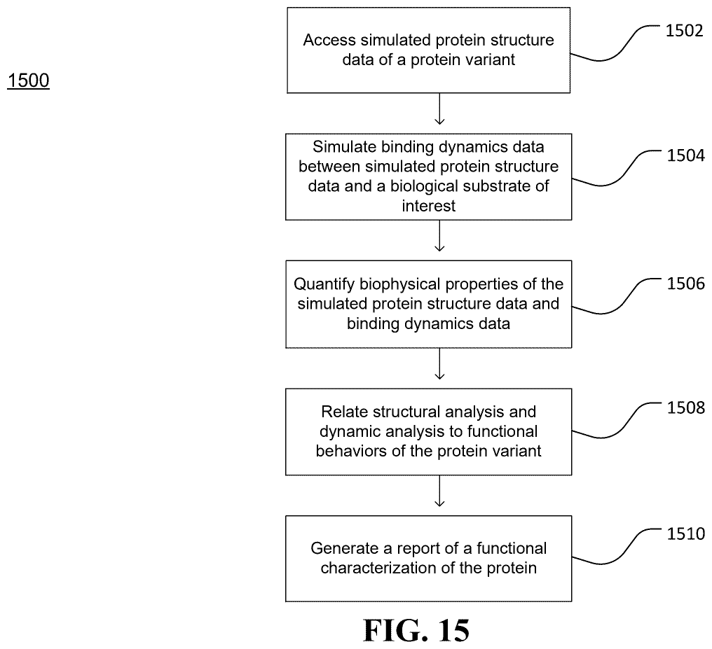

FIG. 15 shows an example process for generating a report that provides a functional characterization of protein residues and/or a protein. At 1502, process 1500 can access simulated protein structure data of a protein variant. The protein structure can be simulated using a computer system. Additionally or alternatively, the computer system can receive or otherwise access previously simulated protein structure data. Accessing the simulated protein structure data may include retrieving such data from a memory or other suitable data storage device or medium. In general, the simulated protein structure data can include simulations of one or more protein structures. In some instances, the simulated protein structure data may include one or more mutations to a base protein structure. At 1504, process 1500 can simulate binding dynamics between the protein and a biological substrate of interest. The biological substrate of interest may be a protein, peptide, ligand, drug, or the like. At 1506, process 1500 can quantify biophysical properties. The biophysical properties may include Tₘ, K_{d}, binding energy, or other biophysical properties. As described above, these biophysical properties can be computed, or otherwise generated, based on the simulated protein structure data. At 1508, process 1500 can relate the structural analysis and dynamic analysis to functional behaviors using the computer system.

Functional behaviors may include flexibility/rigidity and binding affinity. At 1510, process 1500 can generate a report using the computer system. The report may include textual information, quantitative information, data plots, images, models, or other textual, numerical, or visual representations of data that can be presented to a user via the computer system. The report may indicate a functional characterization of specific protein residues, and may indicate a functional characterization of a protein. The functional characterization may include indicating one or more protein residues or domains as especially important in protein binding. The report may further include identifying allosteric sites.

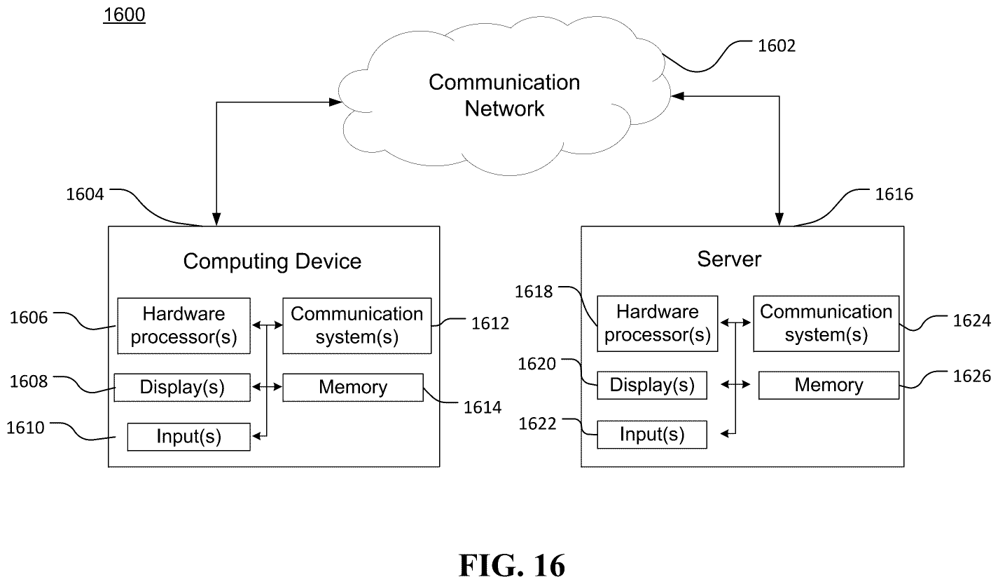

In FIG. 16, an example of a system 1600 (e.g., a data processing system) for characterizing a protein in accordance with some embodiments of the disclosed subject matter is shown.

In some embodiments, computing device 1604 and/or server 1616 can be any suitable computing device or combination of devices, such as a desktop computer, a laptop computer, a smartphone, a tablet computer, a wearable computer, a server computer, a virtual machine being executed by a physical computing device, etc. As described herein, system 1600 can present information about the characterized protein to a user (e.g., a researcher and/or a physician).

In some embodiments, communication network 1602 can be any suitable communication network or combination of communication networks. In some embodiments, communication network 1602 can be any suitable communication network or combination of communication networks. For example, communication network 1602 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 1602 can be a local area network, a wide area network, a public network (e.g., the Internet), a private or semi-private network (e.g., a corporate or university intranet), any other suitable type of network, or any suitable combination of networks. Communications links shown in FIG. 16 can each be any suitable communications link or combination of communications links, such as wired links, fiber optic links, Wi-Fi links, Bluetooth links, cellular links, etc.

FIG. 16 additionally shows an example of hardware that can be used to implement computing device 1604 and server 1616 in accordance with some embodiments of the disclosed subject matter. In some embodiments, computing device 1604 can be used to execute one or more set of instructions to characterize a protein variant, in which the characterization may be functional characterization. In some empirical, computer device 1604 may be used to generate a report based on the functional characterization of a protein variant.

As shown in FIG. 16, computing device 1604 can include one or more hardware processor 1606, one or more displays 1608, one or more inputs 1610, one or more communications 1612, and/or memory 1614. In some embodiments, processor 1606 can be any suitable hardware processor or combination of processors, such as central processing unit, a graphics processing unit, etc. In some embodiments, display 1608 can include any suitable display devices, such as a computer monitor, a touchscreen, a television, etc. In some embodiments, inputs 1610 can include any suitable input device and/or sensors that can be used to receive user input, such as a keyboard, a mouse, a touchscreen, a microphone, etc.

In some embodiments, communication systems 1612 can include any suitable hardware, firmware, and/or software for communicating information over communication network 1602 and/or any other suitable communication networks. For example, communications systems 1612 can include one or more transceivers, one or more communication chips and/or chip sets, etc. In a more particular example, communications systems 1612 can include hardware, firmware and/or software that can be used to establish a Wi-Fi connection, a Bluetooth connection, a cellular connection, an Ethernet connection, etc.

In some embodiments, memory 1614 can include any suitable storage device or devices that can be used to store instructions, values, etc., that can be used, for example, by processor 1606 to present content using display 1608, to communicate with server 1616 via communications system(s) 1612, etc.

Memory 1614 can include any suitable volatile memory, non-volatile memory, storage, or any suitable combination thereof. For example, memory 1614 can include RAM, ROM, EEPROM, one or more flash drives, one or more hard disks, one or more solid state drives, one or more optical drives, etc. In some embodiments, memory 1614 can have encoded thereon a computer program for controlling operation of computing device 1604. In such embodiments, processor 1606 can execute at least a portion of the computer program to present content (e.g., images, user interfaces, graphics, tables, etc.), receive content from server 1616, transmit information to server 1616, etc.

In some embodiments, server 1616 can include a processor 1618, a display 1620, one or more inputs 1622, one or more communications systems 1624, and/or memory 1626. In some embodiments, processor 1618 can be any suitable hardware processor or combination of processors, such as a central processing unit, a graphics processing unit, etc. In some embodiments, display 1620 can include any suitable display devices, such as a computer monitor, a touchscreen, a television, etc. In some embodiments, inputs 1622 can include any suitable input devices and/or sensors that can be used to receive user input, such as a keyboard, a mouse, a touchscreen, a microphone, etc.

In some embodiments, communications systems 1624 can include any suitable hardware, firmware, and/or software for communicating information over communication network 1602 and/or any other suitable communication networks. For example, communications systems 1624 can include one or more transceivers, one or more communication chips and/or chip sets, etc. In a more particular example, communications systems 1624 can include hardware, firmware and/or software that can be used to establish a Wi-Fi connection, a Bluetooth connection, a cellular connection, an Ethernet connection, etc.

In some embodiments, memory 1626 can include any suitable storage device or devices that can be used to store instructions, values, etc., that can be used, for example, by processor 1618 to present content using display 1620, to communicate with one or more computing devices 1604, etc. Memory 1626 can include any suitable volatile memory, non-volatile memory, storage, or any suitable combination thereof. For example, memory 1626 can include RAM, ROM, EEPROM, one or more flash drives, one or more hard disks, one or more solid state drives, one or more optical drives, etc. In some embodiments, memory 1626 can have encoded thereon a server program for controlling operation of server 1616. In such embodiments, processor 1618 can execute at least a portion of the server program to transmit information and/or content (e.g., results of a tissue identification and/or classification, a user interface, etc.) to one or more computing devices 1604, receive information and/or content from one or more computing devices 1604, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

### Numbered Embodiments

Embodiment 1: A method of characterizing a protein variant, the method comprising:
(a) accessing, with a computer system, simulated protein structure data of the protein variant, wherein the simulated protein structure data indicates a structure of the protein variant while unbound;
(b) simulating, using the computer system, binding dynamics data indicating a binding between the simulated protein structure data and a biological substrate of interest;
(c) quantifying, using the computer system, biophysical properties of the simulated protein structure data and binding dynamics data to produce structural analysis and dynamic analysis of the protein variant;
(d) relating, using the computer system, the structural analysis and dynamic analysis to functional behaviors of the protein variant; and
(e) generating, using the computer system, a report based on the functional behaviors, the structural analysis, and the dynamic analysis, wherein the report comprises a functional characterization of the protein variant.

Embodiment 2: The method of embodiment 1, wherein the protein variant comprises a mutation in a domain of interest.

Embodiment 3: The method of any one of embodiments 1 or 2, wherein the biological substrate of interest is a Group I peptide.

Embodiment 4: The method of any one of embodiments 1-3, wherein quantifying the biophysical properties comprises at least one of dynamic flexibility index analysis, adaptive BP-dock analysis, statistical coupling analysis, and principal component analysis, wherein time series of the protein structure, the binding dynamics, or both the protein structure and binding dynamics, may be evaluated.

Embodiment 5: The method of any one of embodiments 1-4, wherein the biophysical properties are selected from a group comprising melting temperature, binding scores, thermostability, and binding assay profile.

Embodiment 6: The method of any one of embodiments 1-5, wherein the method further comprises synthesizing and purifying the protein variant and using experimental methods to further characterize the protein variant.

Embodiment 7: The method of embodiment 6, wherein the experimental methods comprise circular dichroism and isothermal titration calorimetry.

Embodiment 8: The method of any one of embodiments 1-7, wherein the method comprises characterizing a plurality of protein variants.

Embodiment 9: The method of embodiment 8, wherein each protein variant in the plurality of protein variants is ranked based on its performance for a desired function.

Embodiment 10: The method of any one of embodiments 1-9, wherein the functional characterization comprises binding affinity and lowest energy bound poses.

Embodiment 11: The method of any one of embodiments 1-10, wherein the report comprises identifying residues in the protein variant that affect the binding dynamics.

Embodiment 12: A method of designing a protein for a desired function, comprising:
(a) modeling, using a computer system, unbound conformations of a base protein to determine a dominant conformation;
(b) modeling, using the computer system, binding between the base protein in the dominant unbound conformation and a biological substrate of interest to identify a docked pose of the base protein and a contact residue of the base protein, wherein the contact residue of the base protein is in direct contact with the biological substrate;
(c) determining, using the computer system, a flexibility profile of the base protein and modeling dynamics of the base protein;
(d) generating, using the computer system, a plurality of protein variants based on the docked pose, the contact residue, and the flexibility profile of the protein by substituting amino acids at the contact residue;
(e) characterizing, using the computer system, each protein variant in the plurality of protein variants based on structural analysis and dynamic analysis of the protein variant to produce a functional characterization of the protein variant; and
(f) selecting, using the computer system, a protein variant of interest from the plurality of protein variants based on a comparison between the functional characterization of the protein variant and the desired function of the protein variant.

Embodiment 13: The method of embodiment 12, wherein the dynamic analysis of each protein variant in the plurality of protein variants comprises identifying hinges and hinge-shift mechanisms in the protein variant.

Embodiment 14: The method of embodiment 12 or 13, wherein the method further comprises identifying allosteric sites that modulate binding affinity based on the identified hinge-shift mechanisms.

Embodiment 15: The method of any one of embodiments 12-14, wherein the desired function of the protein variant is at least one of flexibility, binding affinity, or lowest energy bound poses.

Embodiment 16: The method of any one of embodiments 12-15, wherein the base protein is based on a WW domain.

Embodiment 17: The method of any one of embodiment 12-16, wherein the biological substrate is selected from a group comprising peptides, proteins, ligands, or drugs.

Embodiment 18: The method of any one of embodiments 12-17, wherein the method further comprises synthesizing and purifying each protein variant in the plurality of protein variants and using experimental methods to further characterize each protein variant.

Embodiment 19: The method of embodiment 18, wherein the experimental methods comprise at least one of circular dichroism or isothermal titration calorimetry.

### REFERENCES

1. Agarwal P K, Billeter S R, Rajagopalan PTR, Benkovic S J, Hammes-Schiffer S. Network of coupled promoting motions in enzyme catalysis. Proc Natl Acad Sci. 2002; 99:2794-9.
2. Aminov R I. A brief history of the antibiotic era: lessons learned and challenges for the future. Front Microbiol. 2010; 1:134.
3. Beach H, Cole R, Gill M L, Loria J P. Conservation of μs-ms enzyme motions in the Apo- and substrate-mimicked state. J Am Chem Soc. 2005; 127:9167-76.
4. Ben Chorin A, Masrati G, Kessel A, Narunsky A, Sprinzak J, Lahav S, et al. ConSurf-DB: an accessible repository for the evolutionary conservation patterns of the majority of PDB proteins. Protein Sci. 2020; 29:258-67.
5. Benkovic S J, Fierke C A, Naylor A M. Insights into enzyme function from studies on mutants of dihydrofolate reductase. Science. 1988; 239:1105-10.
6. Bhabha G, Ekiert D C, Jennewein M, Zmasek C M, Tuttle L M, Kroon G, et al. Divergent evolution of protein conformational dynamics in dihydrofolate reductase. Nat Struct Mol Biol. 2013; 20:1243-9.
7. Bhabha G, Lee J, Ekiert D C, Gam J, Wilson I A, Dyson H J, et al. A dynamic knockout reveals that conformational fluctuations influence the chemical step of enzyme catalysis. Science. 2011; 332:234-8.
8. Boehr D D, McElheny D, Dyson H J, Wright PE. The dynamic energy landscape of Dihydrofolate reductase catalysis. Science. 2006; 313:1638-42.
9. Butler B M, Gerek Z N, Kumar S, Ozkan S B. Conformational dynamics of nonsynonymous variants at protein interfaces reveals disease association. Proteins: structure. Funct Bioinformat. 2015; 83:428-35.
10. Butler B M, Kazan I C, Kumar A, Ozkan S B. Coevolving residues inform protein dynamics profiles and disease susceptibility of nSNVs. PLoS Comput Biol. 2018; 14:e1006626.
11. Cammarata M B, Thyer R, Rosenberg J, Ellington A, Brodbelt J S. Structural characterization of Dihydrofolate reductase complexes by top-down ultraviolet Photodissociation mass spectrometry. J Am Chem Soc. 2015; 137:9128-35.
12. Campitelli P, Modi T, Kumar S, Ozkan S B. The role of conformational dynamics and allostery in modulating protein evolution. Annu Rev Biophys. 2020; 49:267-88.
13. Campitelli P, Ozkan SB. Allostery and epistasis: emergent properties of anisotropic networks. Entropy. 2020; 22:667.
14. Campitelli P, Swint-Kruse L, Ozkan S B. Substitutions at nonconserved rheostat positions modulate function by rewiring long-range, dynamic interactions. Mol Biol Evol. 2021; 38:201-14.
15. Cao H, Wang J, He L, Qi Y, Zhang J Z. DeepDDG: predicting the stability change of protein point mutations using neural networks. J Chem Inf Model. 2019; 59:1508-14.
16. Chakrabarty B, Parekh N. PRIGSA: protein repeat identification by graph spectral analysis. J Bioinform Comput Biol. 2014; 12:1442009.
17. Chakrabarty B, Parekh N. NAPS: network analysis of protein structures. Nucleic Acids Res. 2016; 44:W375-82.
18. Chan H S, Dill K A. Origins of structure in globular proteins. Proc Natl Acad Sci USA. 1990; 87:6388-92.
19. Davies J, Davies D. Origins and evolution of antibiotic resistance. Microbiol Mol Biol Rev. 2010; 74:417-33.
20. del Sol A, Fujihashi H, Amoros D, Nussinov R. Residue centrality, functionally important residues, and active site shape: analysis of enzyme and non-enzyme families. Protein Sci. 2006; 15:2120-8.
21. del Sol A, O'Meara P. Small-world network approach to identify key residues in protein-protein interaction. Proteins: structure. Funct Bioinformat. 2005; 58:672-82.
22. Epstein D M, Benkovic S J, Wright P E. Dynamics of the Dihydrofolate reductase-folate complex: catalytic sites and regions known to undergo conformational change exhibit diverse dynamical features. Biochemistry. 1995; 34:11037-48.
23. Gekko K, Kamiyama T, Ohmae E, Katayanagi K. Single amino acid substitutions in flexible loops can induce large compressibility changes in Dihydrofolate Reductasel. J Biochem. 2000; 128:21-7.
24. Gerek Z N, Kumar S, Ozkan S B. Structural dynamics flexibility informs function and evolution at a proteome scale. Evol Appl. 2013; 6:423-33.
25. Gerek Z N, Ozkan S B. Change in allosteric network affects binding affinities of PDZ domains: analysis through perturbation response scanning. PLoS Comput Biol. 2011; 7:e1002154.
26. Goldenberg 0, Erez E, Nimrod G, Ben-Tal N. The ConSurf-DB: pre-calculated evolutionary conservation profiles of protein structures. Nucleic Acids Res. 2009; 37:D323-7.
27. Hubbard S J, Thornton J M. Naccess. Computer Program; Department of Biochemistry and Molecular Biology: University College London; 1993.
28. Kazan I C, Sharma P, Rahman M I, Bobkov A, Fromme R, Ghirlanda G, Ozkan S B (2022) Design of novel cyanovirin-N variants by modulation of binding dynamics through distal mutations. Faraldo-Gómez J D, Ben-Tal N, editors Elife 11:e67474.
29. Kolbaba-Kartchner B, Kazan I C, Mills J H, Ozkan S B. The role of rigid residues in modulating TEM-1 β-lactamase function and Thermostability. Int J Mol Sci. 2021; 22:2895.
30. Kumar A, Glembo T J, Ozkan S B. The role of conformational dynamics and Allostery in the disease development of human ferritin. Biophys J. 2015; 109:1273-81.
31. Larrimore K E, Kazan I C, Kannan L, Kendle R P, Jamal T, Barcus M, et al. Plant-expressed cocaine hydrolase variants of butyrylcholinesterase exhibit altered allosteric effects of cholinesterase activity and increased inhibitor sensitivity. Sci Rep. 2017; 7:10419.
32. Laxminarayan R, Duse A, Wattal C, Zaidi A K, Wertheim H F, Sumpradit N, et al. Antibiotic resistance—the need for global solutions. Lancet Infect Dis. 2013; 13:1057-98.
33. Luk LYP, Javier Ruiz-Pernia J, Dawson W M, Roca M, Loveridge E J, Glowacki D R, et al. Unraveling the role of protein dynamics in dihydrofolate reductase catalysis. Proc Natl Acad Sci. 2013; 110:16344-9.
34. Maier J A, Martinez C, Kasavajhala K, Wickstrom L, Hauser K E, Simmerling C. ff14SB: improving the accuracy of protein side chain and backbone parameters from ff99SB. J Chem Theory Comput. 2015; 11:3696-713.
35. Martinez J L. Antibiotics and antibiotic resistance genes in natural environments. Science. 2008; 321:365-7.
36. Mauldin R V, Carroll M J, Lee A L. Dynamic dysfunction in Dihydrofolate reductase results from antifolate drug binding: modulation of dynamics within a structural state. Structure. 2009; 17:386-94.
37. Mauldin R V, Lee A L. Nuclear magnetic resonance study of the role of M42 in the solution dynamics of Escherichia coli Dihydrofolate reductase. Biochemistry. 2010; 49:1606-15.
38. McCormick J W, Russo M A, Thompson S, Blevins A, Reynolds K A. Structurally distributed surface sites tune allosteric regulation. Elife. 2021; 10:e68346.
39. Modi T, Campitelli P, Kazan I C, Ozkan S B. Protein folding stability and binding interactions through the lens of evolution: a dynamical perspective. Curr Opin Struct Biol. 2021; 66:207-15.
40. Modi T, Ozkan S B. Mutations utilize dynamic Allostery to confer resistance in TEM-1 β-lactamase. Int J Mol Sci [Internet]. 2018; 19.
41. Modi T, Risso V A, Martinez-Rodriguez S, Gavira J A, Mebrat M D, Van Horn W D, et al. Hinge-shift mechanism as a protein design principle for the evolution of P-lactamases from substrate promiscuity to specificity. Nat Commun. 2021; 12:1852.
42. Ose N J, Butler B M, Kumar A, Kazan I C, Sanderford M, Kumar S, et al. Dynamic coupling of residues within proteins as a mechanistic foundation of many enigmatic pathogenic missense variants. PLoS Comput Biol. 2022; 18:e1010006.
43. Reynolds K A, McLaughlin R N, Ranganathan R. Hot spots for allosteric regulation on protein surfaces. Cell. 2011; 147:1564-75.
44. Rod T H, Radkiewicz J L, Brooks C L. Correlated motion and the effect of distal mutations in dihydrofolate reductase. Proc Natl Acad Sci. 2003; 100:6980-5.
45. Rodrigues J V, Bershtein S, Li A, Lozovsky E R, Hartd D L, Shakhnovich E I. Biophysical principles predict fitness landscapes of drug resistance. Proc Natl Acad Sci. 2016; 113:E1470-8.
46. Salomon-Ferrer R, Gotz A W, Poole D, Le Grand S, Walker R C. Routine microsecond molecular dynamics simulations with AMBER on GPUs. 2. Explicit solvent particle mesh Ewald. J Chem Theory Comput. 2013; 9:3878-88.
47. Sawaya M R, Kraut J. Loop and subdomain movements in the mechanism of Escherichia coli dihydrofolate reductase: crystallographic evidence. Biochemistry. 1997; 36:586-603.
48. Sawle L, Ghosh K. Convergence of molecular dynamics simulation of protein native states: feasibility vs self-consistency dilemma. J Chem Theory Comput. 2016; 12:861-9.
49. Schnell J R, Dyson H J, Wright P E. Structure, dynamics, and catalytic function of dihydrofolate reductase. Annu Rev Biophys Biomol Struct. 2004; 33:119-40.
50. Stevens A O, Kazan IC, Ozkan B, He Y Investigating the allosteric response of the PICK1 PDZ domain to different ligands with all-atom simulations. Protein Sci. 2022; 31:e4474.
51. Tamer Y T, Gaszek I K, Abdizadeh H, Batur T A, Reynolds K A, Atilgan A R, et al. High-order epistasis in catalytic power of Dihydrofolate reductase gives rise to a rugged fitness landscape in the presence of trimethoprim selection. Mol Biol Evol. 2019; 36:1533-50.
52. Thompson S, Zhang Y, Ingle C, Reynolds K A, Kortemme T. Altered expression of a quality control protease in E. coli reshapes the in vivo mutational landscape of a model enzyme. Elife. 2020; 9:e53476.
53. Thorpe IF, Brooks C L III. The coupling of structural fluctuations to hydride transfer in dihydrofolate reductase. Proteins: structure. Funct Bioinformat. 2004; 57:444-57.
54. van den Bedem H, Bhabha G, Yang K, Wright P E, Fraser J S. Automated identification of functional dynamic contact networks from X-ray crystallography. Nat Methods. 2013; 10:896-902.
55. Wang L, Tharp S, Selzer T, Benkovic S J, Kohen A. Effects of a distal mutation on active site chemistry. Biochemistry. 2006; 45:1383-92.
56. Wei Q, Xu Q, Dunbrack R L. Prediction of phenotypes of missense mutations in human proteins from biological assemblies. Proteins. 2013; 81:199-213.
57. Weinreich D M, Delaney N F, DePristo M A, Hartl D L. Darwinian evolution can follow only very few mutational paths to fitter proteins. Science. 2006; 312:111-4.
58. Wong K F, Selzer T, Benkovic S J, Hammes-Schiffer S. Impact of distal mutations on the network of coupled motions correlated to hydride transfer in dihydrofolate reductase. Proc Natl Acad Sci. 2005; 102:6807-12.
59. Koepf, E. K.; Petrassi, H. M.; Sudol, M.; Kelly, J. W. W W: An Isolated Three-Stranded Antiparallel β-Sheet Domain That Unfolds and Refolds Reversibly; Evidence for a Structured Hydrophobic Cluster in Urea and GdnHCl and a Disordered Thermal Unfolded State. Protein Science 2008, 8 (4), 841-853. https://doi.org/10.1110/ps.8.4.841.

1. A method of characterizing a protein variant, the method comprising:
(a) accessing, with a computer system, simulated protein structure data of the protein variant, wherein the simulated protein structure data indicates a structure of the protein variant while unbound;
(b) simulating, using the computer system, binding dynamics data indicating binding between the simulated protein structure data and a biological substrate of interest;
(c) quantifying, using the computer system, biophysical properties of the simulated protein structure data and binding dynamics data to produce structural analysis and dynamic analysis of the protein variant;
(d) relating, using the computer system, the structural analysis and dynamic analysis to functional behaviors of the protein variant; and
(e) generating, using the computer system, a report based on the functional behaviors, the structural analysis, and the dynamic analysis, wherein the report comprises a functional characterization of the protein variant.

2. The method of claim 1, wherein the dynamic analysis comprises identifying hinges and hinge-shift mechanisms in the protein variant.

3. The method of claim 2, wherein the method further comprises identifying allosteric sites that modulate binding affinity based on the identified hinge-shift mechanisms.

4. The method of claim 1, wherein quantifying the biophysical properties comprises at least one of dynamic flexibility index (DFI) analysis, adaptive BP-dock analysis, statistical coupling analysis, and principal component analysis, wherein time series of the protein structure, the binding dynamics, or both the protein structure and binding dynamics, may be evaluated.

5. The method of claim 1, wherein the biophysical properties are selected from a group comprising melting temperature, binding scores, thermostability, and binding assay profile.

6. The method of claim 1, wherein the protein variant is based on a WW domain.

7. The method of claim 1, wherein the protein variant comprises a mutation in an amino acid residue of interest.

8. The method of claim 1, wherein the method further comprises synthesizing and purifying the protein variant and using experimental methods to further characterize the protein variant.

9. The method of claim 8, wherein the experimental methods comprise at least one of circular dichroism and isothermal titration calorimetry.

10. The method of claim 1, wherein the method comprises characterizing a plurality of protein variants, and each protein variant in the plurality of protein variants is ranked based on its performance for a desired function.

11. The method of claim 1, wherein the functional characterization comprises at least one of flexibility, binding affinity, or lowest energy bound poses.

12. The method of claim 1, wherein the report comprises identifying amino acid residues in the protein variant that affect the binding dynamics.

13. A method of designing a protein for a desired function, comprising:
(a) modeling, using a computer system, unbound conformations of a base protein to determine a dominant conformation;
(b) modeling, using the computer system, binding between the base protein in the dominant unbound conformation and a biological substrate of interest to identify a docked pose of the base protein and a contact residue of the base protein, wherein the contact residue of the base protein is in direct contact with the biological substrate;
(c) determining, using the computer system, a flexibility profile of the base protein and modeling dynamics of the base protein;
(d) generating, using the computer system, a plurality of protein variants based on the docked pose, the contact residue, and the flexibility profile of the protein by substituting amino acids at the contact residue;
(e) characterizing, using the computer system, each protein variant in the plurality of protein variants based on structural analysis and dynamic analysis of the protein variant to produce a functional characterization of the protein variant; and
(f) selecting, using the computer system, a protein variant of interest from the plurality of protein variants based on a comparison between the functional characterization of the protein variant and the desired function of the protein variant.

14. The method of claim 13, wherein the dynamic analysis of each protein variant in the plurality of protein variants comprises identifying hinges and hinge-shift mechanisms in the protein variant.

15. The method of claim 14, wherein the method further comprises identifying allosteric sites that modulate binding affinity based on the identified hinge-shift mechanisms.

16. The method of claim 13, wherein the desired function of the protein variant is at least one of flexibility, binding affinity, or lowest energy bound poses.

17. The method of claim 13, wherein the base protein is based on a WW domain.

18. The method of claim 13, wherein the biological substrate is selected from a group comprising peptides, proteins, ligands, or drugs.

19. The method of claim 13, wherein the method further comprises synthesizing and purifying each protein variant in the plurality of protein variants and using experimental methods to further characterize each protein variant.

20. The method of claim 19, wherein the experimental methods comprise at least one of circular dichroism or isothermal titration calorimetry.


SEQUENCE LISTING (7 sequences)

SEQ ID NO: 1 - protein, 35 aa, synthetic construct
GSVESDWSVHTNEKGTPYYHNRVTKQTSWIKPDVL

SEQ ID NO: 2 - protein, 35 aa, synthetic construct
GGSKSGWSEYHNDAGTPYYYDRQTGQSTWEKPEDF

SEQ ID NO: 3 - protein, 35 aa, synthetic construct
GSVESDWSVYTNEKGTPYYHNRVTKQTSWIKPDVL

SEQ ID NO: 4 - protein, 35 aa, synthetic construct
GSVESDWSVHTNEKGTPYYYNRVTKQTSWIKPDVL

SEQ ID NO: 5 - protein, 35 aa, synthetic construct
GSVESDWSVYTNEKGTPYYYNRVTKQTSWIKPDVL

SEQ ID NO: 6 - protein, 35 aa, synthetic construct
GSVESDWSVHHNEKGTPYYHNRVTKQTSWIKPDVL

SEQ ID NO: 7 - protein, 13 aa, synthetic construct
EYPPYPPPPYPSG